(19) United States
(12) Patent Application Publication
(10) Pub. No.: US 2009/0305317 A1
(43) Pub. Date: Dec. 10, 2009

(54) USER INTERFACE FOR TESTING DEVICE

(76) Inventors: Jacob S. Brauer, Chicago, IL (US); Darren Brown, Harrison, NY (US); Kevin Chang, Granger, IN (US); Jun Chen, Warren, NJ (US); Richard Kates, Ridgewood, NJ (US); Jennifer M. Levin, Chicago, IL (US); Harris Lieber, White Plains, NY (US); Jing Lin, Granger, IN (US); Dawn Rountree, Derby, CT (US); Amy Schwartz, Evanston, IL (US); Fred Sommer, Ardsley, NY (US); Fu Hsiung Tsai, Mishawaka, IN (US); Huan-Ping Wu, Granger, IN (US); Raymond Yao, Tarrytown, NY (US)

Correspondence Address: John C. Gatz;Nixon Peabody LLP, 161 North Clark Strret, 48th Floor, Chicago, IL, 60601-3213, US

(21) Appl. No.: 12/468,671

(22) Filed: May 19, 2009

Related U.S. Application Data
(60) Provisional application No. 61/059,244, filed on Jun. 5, 2008.

(51) Int. Cl. C12Q 1/54; C12M 1/34

(52) U.S. Cl. 435/14; 435/287.1

(57) ABSTRACT

A testing system for testing an analyte in a fluid sample includes a user interface including a display for displaying information relating to measurements of health data and an input device for receiving information from a user relating to the health data. The testing system further includes an automarking feature adapted to identify a testing result of a control solution, the testing of the control solution being distinguishable from the testing of the fluid sample. The testing result of the control solution is not included in the information relating to the measurements of health data that is displayed to a user via the user interface.

### CROSS-REFERENCE To RELATED APPLICATION

This application claims the benefit of U.S. Provisional Patent Application No. 61/059,244, filed Jun. 5, 2008, which is hereby incorporated by reference in its entirety.

### FIELD OF THE INVENTION

The present invention relates generally to systems and methods for the testing and monitoring of health data. More specifically, the systems and methods of the present invention provide an interface for displaying information regarding the testing and monitoring of health data in a more useful and accurate manner.

### BACKGROUND OF THE INVENTION

The quantitative determination of analytes in body fluids is of great importance in the diagnoses and maintenance of certain physiological conditions. For example lactate, cholesterol and bilirubin should be monitored in certain individuals. In particular, it is important that individuals with diabetes frequently check the glucose level in their body fluids to regulate the glucose intake in their diets. The results of such tests can be used to determine what, if any, insulin or other medication needs to be administered.

Diagnostic systems, such as blood-glucose systems, include a meter or instrument used to calculate a glucose value based on a measured output, such as current or color, and the known reactivity of the reagent-sensing element used to perform the test. Blood-glucose systems typically allow a user to collect a blood sample on a test sensor in which the test sensor is located in the meter. The meter measures the reaction between the glucose in the blood sample and a reagent from the test sensor to determine the blood-glucose concentration in the sample. These systems may store test results in the meter and may display the results to the user. A keypad or other interactive component may also be provided on a meter to allow a user to access the test results.

To obtain more accurate measurements, control solutions containing known amounts of glucose are used to verify that the instrument is operating properly. Control solutions are used to check the functionality of the analyte monitoring device or meter. Control solutions need to be identified and separated from the readings of real whole blood samples. Specifically, there is a need to automatically detect the control solution by the meter for several reasons. First, the temperature coefficients of the control solution and whole blood may be different. Thus, it is desirable to compensate the temperature effect on glucose readings with separate temperature coefficients. Second, by automatically detecting the control solution and not recording its reading into the memory of real blood-glucose readings assists to provide a more accurate average of blood-glucose readings. Without eliminating the control-solution readings, control solutions will be included in the history of the glucose measurements. Having incorrect historical readings may lead to an incorrect interpretation of a patient's diabetic condition. Additionally, if a control solution is substituted for a whole blood sample, it may be erroneously considered by a physician as indicating a need to change treatment. Third, automatically detecting the control solution and not recording its reading into the memory of blood-glucose readings may minimize the chance of faking the blood-glucose readings by control solution.

Therefore, it would be desirable to have a feature for automatically detecting or marking control-solution readings and to separate the control-solution readings from the testing data of the real whole blood samples.

### SUMMARY OF THE INVENTION

According to one embodiment, a testing system for testing an analyte in a fluid sample comprises a user interface including a display for displaying information relating to measurements of health data and an input device for receiving information from a user relating to the health data. The testing system further includes an automarking feature adapted to identify a testing result of a control solution, the testing of the control solution being distinguishable from the testing of the fluid sample, and wherein the testing result of the control solution is not included in the information relating to the measurements of health data that is displayed to a user.

### BRIEF DESCRIPTION OF THE DRAWINGS

FIG. 1A illustrates a testing system having an interface for displaying health data.

FIG. 1B illustrates the testing system of FIG. 1A showing a user interface displaying a control solution test reading according to one embodiment.

FIG. 1C illustrates the testing system of FIG. 1A showing a user interface displaying a logbook feature having a control solution test reading according to another embodiment.

FIG. 1D illustrates the testing system of FIG. 1A showing a user interface displaying a control solution test reading according to a further embodiment.

While the invention is susceptible to various modifications and alternative forms, specific embodiments are shown by way of example in the drawings and are described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATED EMBODIMENTS

The present invention is directed to a testing system that provides information relating to health data. This health data may be collected, measured or input by a user. One example of such health data is an analyte concentration in a body fluid sample, such as glucose in a blood sample. Other types of health data may include heart rate measurements, blood pressure measurements, body temperature measurements, breathing measurements for chronic obstructive pulmonary disease (COPD) analysis, weight measurements for analyzing Lasix use, and the like. For measurements that do not require analyte testing, the testing device 10 may monitor and analyze these types of health data and provide a user with the relevant information about the user's medical condition. Wherein the following description refers mainly to testing of analytes in fluid samples, it will be appreciated that other types of health data may be used with aspects of the present invention.

In some embodiments, a testing device as described herein may be employed in a larger health data management system that connects the testing device with other external processing devices, health care devices, and/or other devices/systems. The testing device may take advantage of the processing and user interface capabilities of such devices. For example, some functionalities may be better viewed on external processing devices if the size of the user interface on the testing device is too compact. Meanwhile, the health care devices may take advantage of the processing and user interface capabilities of the testing device. The interface between the testing device and the external devices may employ a wired communication protocol, such as the universal serial bus (USB) standard, or a wireless communication protocol, such as Bluetooth® technology.

For example, the testing device may be a blood glucose meter that interfaces with a processing device, such as a conventional personal computer (PC). Although the blood glucose meter may include advanced data processing and display features as described herein, users of the blood glucose meter may access more sophisticated analyses and presentations of blood glucose test data by connecting the blood glucose meter to a processing device that executes data-management software. For example, the software may be a product similar to WINGLUCOFACTS® Diabetes Management Software available from Bayer HealthCare LLC (Tarrytown, N.Y.). In another example, the testing device may be a blood glucose meter that interfaces with a health care device, such as a heart rate monitor, that transmits health data that can be combined with the data collected by the blood glucose meter itself.

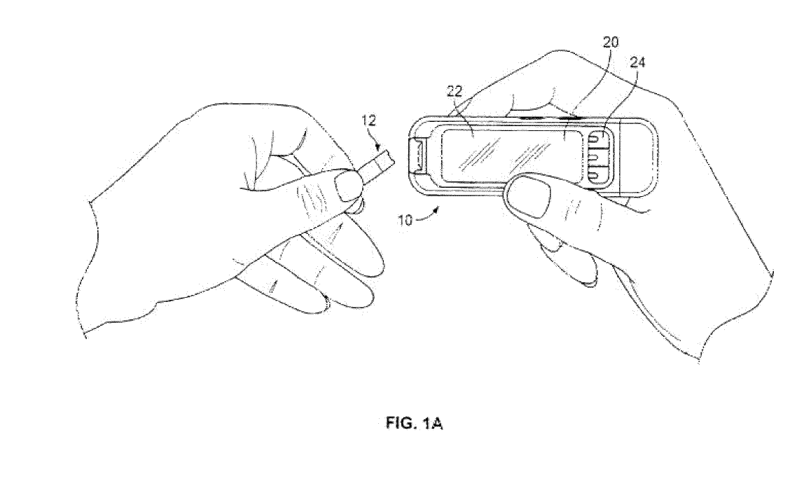

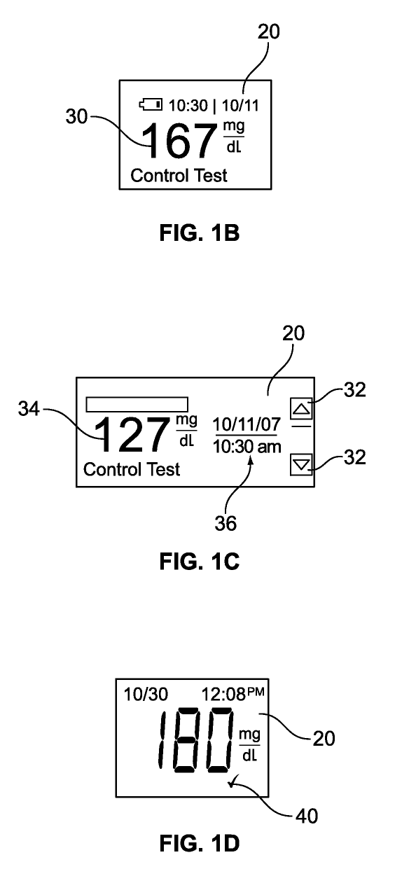

Referring to FIG. 1A, one embodiment of a testing device 10 and a test sensor 12 is illustrated. The test sensor 12 is configured to receive a fluid sample which is analyzed using the testing device 10. Analytes that may be analyzed include glucose, lipid profiles (e.g., cholesterol, triglycerides, LDL and HDL), microalbumin, hemoglobin Al_{C}, fructose, lactate, or bilirubin. The analytes may be in, for example, a whole blood sample, a blood serum sample, a blood plasma sample, other body fluids like ISF (interstitial fluid) and urine, and non-body fluids.

The test sensor 12 may include a fluid-receiving area (not shown). The fluid-receiving area contains a reagent which reacts with a fluid sample to indicate the concentration of an analyte in the fluid sample. For example, the fluid-receiving area may receive a fluid sample, such as a blood sample. The fluid-receiving area may also receive a liquid control solution. The liquid control solution contains a control marker (also referred to as in internal reference). The control marker is configured to generate a distinctive current profile using a detection algorithm. By having a distinctive current profile, the testing device 10 can automatically distinguish a control test from an analyte-fluid test (e.g., a glucose blood sample).

The control marker may be used in an electrochemical test sensor that is adapted to assist in determining information related to an analyte, such as an analyte concentration. The electrochemical test sensor typically includes a plurality of electrodes and a fluid-receiving area that contains an enzyme. The fluid-receiving area includes a reagent for converting an analyte of interest (e.g., glucose) in a fluid sample (e.g., blood) into a chemical species that is electrochemically measurable, in terms of the electrical current it produces, by the components of the electrode pattern. The reagent typically contains an enzyme such as, for example, glucose oxidase, which reacts with the analyte and with an electron acceptor such as a ferricyanide salt to produce an electrochemically measurable species that can be detected by the electrodes. It is contemplated that other enzymes may be used to react with glucose such as glucose dehydrogenase. In general, the enzyme is selected to react with the desired analyte or analytes to be tested so as to assist in determining an analyte concentration of a fluid sample. If the concentration of another analyte is to be determined, an appropriate enzyme is selected to react with the analyte.

The reagent also typically includes a mediator that assists in transferring electrons between the analyte and the electrodes. The reagent may include binders that hold the enzyme and mediator together, other inert ingredients, buffers or combinations thereof.

The testing device 10 includes a reaction-detection system for measuring the concentration of analyte for the sample collected by the test sensor 12. As described above, the reaction-detection system may include contacts for the electrodes to detect the electrochemical reaction for an electrochemical test sensor. Alternatively, the reaction-detection system may include an optical detector to detect the chromatic reaction for an optical test sensor. To calculate the actual concentration of analyte from the electrochemical or chromatic reaction measured by the reaction-detection system and to generally control the procedure for testing the sample, the testing device 10 employs at least one processor (not shown), which typically executes programmed instructions according to a measurement algorithm. Data processed by the processor may be stored in a memory element.

The testing device 10 of FIG. 1A includes a user interface 20, which includes a display 22 and a user input device 24. The display 22 typically displays information regarding the test results, the testing procedure and/or information in response to signals input by the user, including text and images. The display 22 may be a graphic liquid crystal display (LCD), an organic light-emitting diode (OLED), segment LCD, or the like. The user input device 24 allows the user to interact with the testing device 10 and may include pushbuttons, soft keys, a scroll wheel, touch screen elements, or any combination thereof.

It is contemplated that the user interface 20 may provide a high-resolution, rich viewing display 22, which may present both static and moving text and images to the user. However, other types of displays, including, for example, lower resolution, monochromatic LCD displays, may be employed. In general, a range of display types, from a low-cost basic display to a fully functional display, may be employed. The display 22 may be of any suitable size. In some cases, the display 22 may cover one entire side of the testing device 10. Moreover, the display 22 may include a touchscreen. In addition, the user interface 20 may provide advanced graphical user display and audio capabilities available directly on the testing device 10 or via a communications interface with the testing device 10.

As described previously, the testing device 10 employs at least one processor that typically executes programmed instructions, as well as the user interface 20, which includes the display 22 to present information to the user, and input devices 24, such as pushbuttons, soft keys, a scroll wheel, touch screen elements, or any combination thereof, to enable interaction with the user. With such components, the testing device 10 generally controls the procedure for testing the sample and calculating the test results and for providing a plurality of user features. Certain of the user features of the testing device 10 may be available to the user via a hierarchical menu. The user is allowed to navigate through the hierarchical menu to access certain features of the testing device 10 that are described in more detail below. In some embodiments, the hierarchical menu has no more than four levels to provide quick and convenient access to the features of the device. For example, a user may operate a set of soft keys that corresponds to items in the hierarchical menu. In one embodiment, the testing device 10 provides three soft keys that are not dedicated to specific functions. Rather, the display 22 shows one set of three menu items and each of the soft keys is assigned to one of the menu items. Operating a soft key selects the corresponding menu item and either navigates the user to another level in the hierarchical menu or executes a particular function. Because the menu items are dynamically assigned to the soft keys, the user interface 20 does not require a separate key for each possible function, so many different functions are available even in a compact user interface 20. Further examples of such soft keys are described in detail herein below.

In some embodiments, to provide an easier and more intuitive process of entering information, the user interface 20 may prompt the user to input information or instructions into the testing device 10 relating to one or more features. More specifically, the user may be asked to respond to simple prompts or make menu selections to guide the user during operation of the testing device 10. For example, the user may be prompted to enter information relating to an autologging feature. An autologging features allows information to be received by the test device 10 to enhance the output of information to the user.

As discussed above, according to one embodiment of the present invention, it is highly desirable for a control solution to provide accurate analyte readings and be distinguishable from a biological sample. The present invention employs an oxidizable species (i.e., a control marker) that is oxidizable only at voltages higher than those used for the analyte (e.g., glucose) measurements. This means that at a low potential adequate to fully oxidize the analyte-related mediator, but not the control marker, only the analyte will be measured. The term control marker is also referred to as an internal reference. When the potential is high enough to oxidize the added control marker, both the analyte and the control marker will be oxidized. Although the analyte (e.g., glucose) is oxidized at the higher potential, the measurement made at a lower voltage is already diffusion-limited and does not depend on the total amount of analyte oxidized by the enzyme. It is feasible, therefore, to add such control markers to a control solution and to use it to identify the solution as a control and not as a biological sample.

The control markers to be used include the following: sodium iodide, triethanolamine, tripropanolamine, tributanolamine, 2,5-dihydroxybenzoic acid, xylenol orange, hydroquinone sulfonic acid or cresol red (C₂₁H₁₇NaO₅S). In one method, the sodium iodide may be used in combination with a phenothiazine mediator or phenoxazine mediator such as, for example, 3-(2′,5′-disulfophenylimino)-3H-phenothiazine mediator. It is also contemplated that the control markers of 2,5-dihydroxybenzoic acid, xylenol orange, hydroquinone sulfonic acid and cresol red may also be used with a phenothiazine mediator or phenoxazine mediator such as, for example, 3-(2′,5′-disulfophenylimino)-3H-phenothiazine. In one method, the triethanolamine may be used in combination with a ferricyanide-based mediator such as potassium ferricyanide. It also contemplated that the control marker of tripropanolamine and tributanolamine may be used in combination with a ferricyanide-based mediator such as potassium ferricyanide. It is contemplated that the above-identified controls makers may be used with other mediators.

The difference between the currents measured at high and low voltages may be compared to indicate the presence of the internal reference characteristic of the control solution. In one non-limiting method, a Differential Index (DI) may be employed following current components relating to the analtye (e.g., glucose) and the control marker:

DI=i_{high volt/}i_{low volt}=(i_{int ref}+i_{glucose})/_{glucose}=1+i_{int ref}/i_{glucose}

where i_{high volt} is the current measured at the higher voltage
i_{low volt} is the current measured at the lower voltage

It follows that if the control marker is not present (such as in the blood samples), i_{int ref} should be zero and the i_{high volt} will be substantially the same as i_{low volt}. Thus, the DI value will typically approach 1 when the control marker is not present. The DI value in practice, however, may have values over 1 when the control marker is not present, especially when a lower glucose concentration is measured during a change from a low voltage to a higher voltage. In such a scenario, the control marker may have a higher DI than 1.

When the control marker is present, the value of DI will be greater than 1, depending on the amount of the control marker relative to the amount of analyte. If the amount of control marker added to the control solution provides a current similar to that from oxidizing the analyte-related mediator, the DI value may be generally about two times that from oxidizing the analyte-related mediator. The control marker may be included in an amount suitable for control solutions corresponding to a high analyte concentration.

It is typical to use several control solutions corresponding to low, normal, and high analyte concentration to test a glucose meter. If, for example, the amount of the control marker is chosen so that the DI value is 1.75 or greater for the highest analyte concentration in the control solution, the current from the control marker will be relatively large compared to the current for the analyte in the lowest analyte control solution. Then the same amount of the control marker used with a control solution having a low analyte concentration will provide an even higher value of DI. Such high DI values will provide higher confidence in the presence of a control solution, rather than a biological sample (e.g., whole blood). It is contemplated that other methods for determining the presence of the control marker in the control solution may be used with the present invention.

Referring back to the user interface 20, upon applying a fluid sample to the test sensor 12, the user may be prompted to enter information into the testing device 10 relating to the fluid sample. To enter the requested information, the user may select from one or more user-selectable options displayed on the user interface 20. The user-selectable options may displayed adjacent to one or more input devices 24, such as soft keys, for receiving the user's input. In another example, the input devices 24 may also be used to retrieve information, such as test results, and to present the information on the display 12.

For the case of a fluid sample that is a control solution, the user may not have to enter information that identifies the fluid sample as a control sample because, as discussed above, the testing device 10 is able to detect the presence of the control marker. An example of a user interface displaying the results of the control solution test are shown in FIG. 1B. From this screen, the user can view the concentration 30 of the control test, and note that it is labeled “control test.” In addition, the date and time of the testing of the control solution may be displayed. These results may then be saved in the memory of the testing device 10.

Under some testing features of the testing device 10, the user interface 20 prompts the user to press the input device 24 to select from a set of user-selectable options 30 that correspond to the fluid sample being tested. Such information may be provided by inputting a single “click” of one of the soft keys on the input device 24. The particular user-selectable options may include indicators, such as meal markers, that indicate when the fluid sample was taken in relation to when a meal has or has not been eaten. For example, one set of meal markers may include a “before food” marker, an “after food” marker and a “skip” or “none” marker. It is also contemplated that, even though the detection of a control marker will happen automatically, the user may also be able to select a “control” indicator when prompted for information relating to the testing sample.

In the embodiment shown in FIG. 1C, the user interface 20 displays a logbook function. Using scroll keys 32, the user can scroll through test results, including control solution test results, to view a concentration reading 34 of an earlier control solution test. The user interface 20 may also display the date and time 36 of the control solution test. Thus, the user can review the last time that a control solution test was performed. Instead of using text to indicate that a reading is a control solution test reading, an icon 40 may be used, such as a “check” mark or other such mark, as shown in FIG. 1C. This icon indicates to the user that the reading is a based on results of a control solution test and not a blood sample test, for example. The log book feature may also allow the user to review dates, times and readings of prior concentration values in blood samples. Such a feature in effect automates the task of keeping a paper logbook by most individual with diabetes and also helps healthcare providers to draw their patients' attention to how food affects blood glucose readings.

In some embodiments, the information that is provided by the user may be categorized so that an evaluation of the data yields a more useful analysis for the user. Categorizing health data helps the user to gain a better understanding of what values are being averaged and makes the data more actionable. In some embodiments, the categorization of information may be customized for different user groups, such as children or the elderly. Such categorization may be useful, for example, when taking averages of test results as certain averages, without more specific indicators, can mask information that may be useful in treating a disease.

As mentioned above, it is particularly important when displaying the averaging information that such averages do not include the testing results of the control solution test. By excluding the control solution test results, the average of the testing result will not include control solution readings that may lead to an incorrect interpretation of a patient's diabetic condition. For example, certain averages may be selected by the user, from a list of selectable averages, such as “7-day” average, a “14-day” and a “30-day.” By automatically identifying a control solution testing result, the control solution testing result will not be substituted for a whole blood sample, and thus will not be erroneously considered by a physician as indicating a need to change treatment.

Furthermore, the user interface 20 may also provide information regarding target ranges for certain categories of readings, for example, a pre-meal target range and a post-meal target range. These embodiments may reveal important information about the components of the average reading, such as whether the average reading is above a target range, below a target range or within a target range. This useful information may also indicate the number of readings that fall within the target range, the number of readings that fall above the target range, and the number of readings that fall below the target range. Also, the total number of readings that are used to provide the average value may be displayed for each of the specific averaging readings. Such features, which indicate the number of readings within and outside of a target range, provides useful information to the user, as well as a physician or nurse, to better reveal the trend of readings and to spot potentially troubling readings which a user may want to address. Thus, it would be problematic if the numbers of readings and the averages included erroneous testing data due to control solution testing results.

In some embodiments, the user interface 20 may also allow users to further investigate the average reading and view the memory for more specific readings composing the average readings contained in a log book function. This way the user may be able to confirm that no control solution results are included in averages displayed to the user. In general, the aspects of the embodiments described herein help to assure the user and healthcare professionals that no unwanted data is included in the averages, the numbers of concentration readings above, below or within target zones, etc.

Other types of information may be entered by a user to add additional notes regarding the health data. For example, a user may be able to enter such notes as “gym day,” “sick,” “stress,” “activity,” “don't feel right,” “traveling” and the like, to further identify the factors that may affect the measurement of the health data. Such labeling provides important information about lifestyle factors that enhance the value of the data to the users. Predefined notes may be provided for convenience, or the user may be able to customize notes through the user interface 20. In other embodiments, the user may create notes through a separate software system and upload the notes to the testing device 10 through a communication interface.

In general, the embodiments described herein provide features for automatically detecting or marking control-solution readings and for separating the control solution readings from the testing data of the whole blood samples. Thus, the user can be assured that the health data that is being displayed via the user interface is an accurate determination of his or her condition. This is particularly advantageous as the user is not required to input any additional information to account for control solution readings and thus the possibility of a user failing to account for a control solution testing result is reduced or eliminated. If desired, however, the user interface may still access information pertaining to the control solution testing that is available through the logbook and autologging features.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention.

What is claimed is:

1. A testing system for testing an analyte in a fluid sample, comprising:
a user interface including a display for displaying information relating to measurements of health data and an input device for receiving information from a user relating to the health data; and
an automarking feature adapted to identify a testing result of a control solution, the testing of the control solution being distinguishable from the testing of the fluid sample, and wherein the testing result of the control solution is not included in the information relating to the measurements of health data that is displayed to a user via the user interface.

2. The testing system of claim 1, wherein the automarking feature identifies the testing result of the control solution due to the presence of a control marker in the control solution.

3. The testing system of claim 2, wherein the control marker is an oxidizable species that is oxidizable only at voltages higher than those used for the analyte measurements.

4. The testing system of claim 1, wherein the user interface displays the result of the control solution testing.

5. The testing system of claim 4, wherein the user interface includes an icon for indicating a control solution testing result.

6. A method of testing an analyte in a fluid sample, the method comprising the acts of:
identifying a testing result of a control solution using an automarking feature;
distinguishing the testing result of a control solution from a testing result of the fluid sample; and
displaying information relating to measurements of health data on a user interface, the testing result of the control solution not being included in the information relating to the measurements of health data.

7. The method of claim 6, further comprising receiving information from a user relating to health data via a user interface.

8. The method of claim 6, wherein the act of identifying the testing result of the control solution includes detecting the presence of a control marker in the control solution.

9. The method of claim 8, wherein the control marker is an oxidizable species that is oxidizable only at voltages higher than those used for the analyte measurements.

10. The method of claim 1, further comprising displaying the result of the control solution testing via the user interface.

11. The method of claim 10, wherein the user interface includes an icon for indicating a control solution testing result.
